(19) United States
(12) Patent Application Publication
(10) Pub. No.: US 2016/0009616 A1
(43) Pub. Date: Jan. 14, 2016

(54) Method for Producing a Water Soluble Menthol Compounds having Antibacterial, Anti-Inflammatory, and Bacteriostatic Effects

(71) Applicant: Wen Hsiang Wayne Hsin, Palos Verdes, CA (US)

(72) Inventor: Wen Hsiang Wayne Hsin, Palos Verdes, CA (US)

(21) Appl. No.: 14/328,464

(22) Filed: Jul. 10, 2014

(51) Int. Cl. C07C 29/70

(52) U.S. Cl. CPC C07C 29/70; C07C 2101/14

(57) ABSTRACT

A method for producing water soluble menthol compound produces a menthol compound that is soluble in alcohol and ether, yet insoluble in water. The menthol compound provides antibacterial, anti-inflammatory, and bacteriostatic effects when combined with other compounds to produce numerous medical and consumable compositions, including, without limitation, eye drops, mouth wash, juices, and noodles. In some embodiments, the produced menthol compound is a menthol water-soluble sodium salt or sylvite. However in other embodiments, the menthol compound may include a menthol crystal configuration. The steps include: adding a metal to a menthol solution. Enabling the reaction; dissolving the solid mixture; applying osmosis membrane dialysis to the dissolved solid mixture; filtering, drying, and washing the solid mixture; decompressing and removing water from the solid mixture; and collecting the water soluble menthol compound.

### BACKGROUND OF THE INVENTION

1. Field of the Invention

This invention relates to a method for producing menthol water soluble compounds, and more particularly relates to a method for producing a water soluble menthol salt that has antibacterial effects when combined with other compounds for use in medicines, foods, and beverages.

2. Description of the Related Art

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

It is commonly known that menthol is an organic compound made synthetically or obtained from cornmint, peppermint, or other mint oils. It is a waxy, crystalline substance, clear or white in color, which is solid at room temperature and melts slightly above. The main form of menthol occurring in nature is (−)-menthol, which is assigned the (1R,2S,5R) configuration. Menthol has local anesthetic and counterirritant qualities, and it is widely used to relieve minor throat irritation.

Often, menthol is manufactured as a single enantiomer (94% ee). The process involves an asymmetric synthesis. The process begins by forming an allylic amine from myrcene, which undergoes asymmetric isomerisation in the presence of a BINAP rhodium complex to give (after hydrolysis) enantiomerically pure R-citronellal. This is cyclised by a carbonyl-ene-reaction initiated by zinc bromide to isopulegol, which is then hydrogenated to give pure (1R,2S,5R)-menthol.

It is widely known that another commercial process for producing menthol is the Haarmann-Reimer process. This process starts from m-cresol which is alkylated with propene to thymol. This compound is hydrogenated in the next step. Racemic menthol is isolated by fractional distillation. The enantiomers are separated by chiral resolution in reaction with methyl benzoate, selective crystallization followed by hydrolysis.

Typically, menthol, is an important substance widely used in the field of, for example, food additives, drug components, cosmetics, fragrances and medicines. I-Menthol is the main component of the mentha oils from Mentha an/ensis and Mentha piperita. I-Menthol is generally obtained from the crude mentha oil by crystallization. Depending on the crystallization method and the starting material, the crystals differ in terms of taste, size and shape. Residual liquid mentha oil adhering to the menthol crystals obtained from mentha oils affects the sensory profile of the crystals. I-Menthol has conventionally been used as a flavor for food, including sweets and oral refreshments such as, for example, chewing gum, candy, cigarettes and the like. The presence of trace amounts of impurities can detrimentally affect the quality and flavor of l-menthol, and therefore, processes for producing highly pure 1-menthol have been of interest for a long time.

It is known that bacteria constitute a large domain of prokaryotic microorganisms. Typically a few micrometers in length, bacteria have a number of shapes, ranging from spheres to rods and spirals. Bacteria were among the first life forms to appear on Earth, and are present in most of its habitats. Bacteria inhabit soil, water, acidic hot springs, radioactive waste, and the deep portions of Earth's crust.

Bacteria also live in symbiotic and parasitic relationships with plants and animals. Specifically, the mouth harbors a diverse, abundant and complex microbial community. This highly diverse microflora inhabits the various surfaces of the normal mouth. Bacteria accumulate on both the hard and soft oral tissues in biofilms. Bacterial adhesion is particularly important for oral bacteria.

A bacteriostatic agent or bacteriostatic is a biological or chemical agent that stops bacteria from reproducing, while not necessarily harming them otherwise. Depending on their application, bacteriostatic antibiotics, disinfectants, antiseptics and preservatives can be distinguished. Upon removal of the bacteriostatic, the bacteria usually start to grow again. The inhibition of bacteria in medicines, foods, and beverages is important. This is better served when combined with a pleasant flavor in the medicine, food, or beverage applied to inhibit the bacteria.

In view of the foregoing, it is clear that these traditional techniques are not perfect and leave room for more optimal approaches.

### SUMMARY OF THE INVENTION

From the foregoing discussion, it should be apparent that a need exists for a method for producing menthol water soluble compounds. The method for producing water soluble menthol compound produces a menthol compound that is soluble in alcohol and ether, yet insoluble in water. The menthol compound provides antibacterial, anti-inflammatory, and bacteriostatic effects when combined with other compounds to produce numerous medical and consumable compositions, including, without limitation, eye drops, mouth wash, juices, and noodles. In some embodiments, the produced menthol compound is a menthol water-soluble sodium salt or sylvite. However in other embodiments, the menthol compound may include a menthol crystal configuration.

Those skilled in the art will recognize that menthol crystals are known in the art to be a main ingredient in mint and peppermint oil. The produced menthol compound may include an organic compound derived from mentha arvensis oil, and extracted from mint leaves and stems. In some embodiments, the menthol compound may be used in the preparation of perfumes, cosmetics, analgesic balms, ointments, lotions, creams, tooth paste, shampoos and conditioners. In the present invention, at least four different variations of the method may be derived and combined with additional compounds. The uses may include, without limitation, eye drops, mouth wash, juices, and noodles. In any case, the menthol compound creates antibacterial, anti-inflammatory, and bacteriostatic effects on the medicines, foods, and beverages.

In one embodiment, the menthol compound comprises a molecular formula of C₁₀H₂₀O. The final menthol compound is collected in molten or lump form. In some embodiments, the menthol crystals may appear colorless, prism-shaped, or needle shaped. In one embodiment, the produced menthol compound is a white color crystal. The crystals are generally solid at about 42° Celsius, melting at a temperature slightly above 43° Celsius. Additionally, the method may produce various isomers of menthol, including, without limitation, menthol, D-menthol, L-menthol, DL-menthol, and racementhol.

The method of producing the menthol compound, and derivatives thereof, comprises standard wet chemistry and uses instrumentation and methods known in art, but combined in a novel manner. The method also utilizes both quantitative and qualitative chemical measurements. In one embodiment, a first Step for producing the menthol compound may include adding a metal to a menthol solution. A second Step comprises enabling a reaction between the metal and the menthol alcohol solution. The method may further include a Step of collecting a solid mixture formed by the reaction. A Step further comprises dissolving the solid mixture. Additionally, a Step may include applying osmosis membrane dialysis to the dissolved mixture. The method may also include a Step of preparing the solid mixture for collection. The preparation may include decompressing the solid mixture and removing water from the solid mixture. A final Step comprises collecting the water soluble menthol compound.

One objective of the present invention is to produce a menthol compound that has antibacterial, anti-inflammatory, and bacteriostatic effects on medicines, foods, and beverages.

Another objective is to produce a menthol compound that is soluble in alcohol and ether, yet insoluble in water.

Another objective is to derive a menthol compound that can be combined with other compounds produce an antimicrobial agent partially from the menthol compound that inhibits bacteria from reproducing, while not necessarily harming the bacteria otherwise.

Yet another objective is to derive a menthol compound that can be combined with other compounds to produce eye drops that are effective for soothing itch and irritation.

Yet another objective is to derive a menthol compound that can be combined with other compounds to produce flavoring for beverages and foods.

Yet another objective is to produce a menthol compound that can be orally and topically administered to help relieve medical ailments.

Yet another objective is to produce a menthol compound that provides flavoring to food and beverages.

Yet another objective is to produce a menthol compound inexpensively, using standard wet chemistry techniques and instrumentation.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a more particular description of the invention will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 illustrates a flowchart diagram of an exemplary method for producing water soluble menthol, in accordance with an embodiment of the present invention;

FIG. 2 illustrates a structural formulas for isomers of a menthol compound isomer, a D-menthol isomer, and an L-menthol isomer, in accordance with an embodiment of the present invention; and

FIG. 3 illustrates a table demonstrating an antibacterial and bacteriostatic efficacy exam against different types of bacteria, in accordance with an embodiment of the present invention.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are provided. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

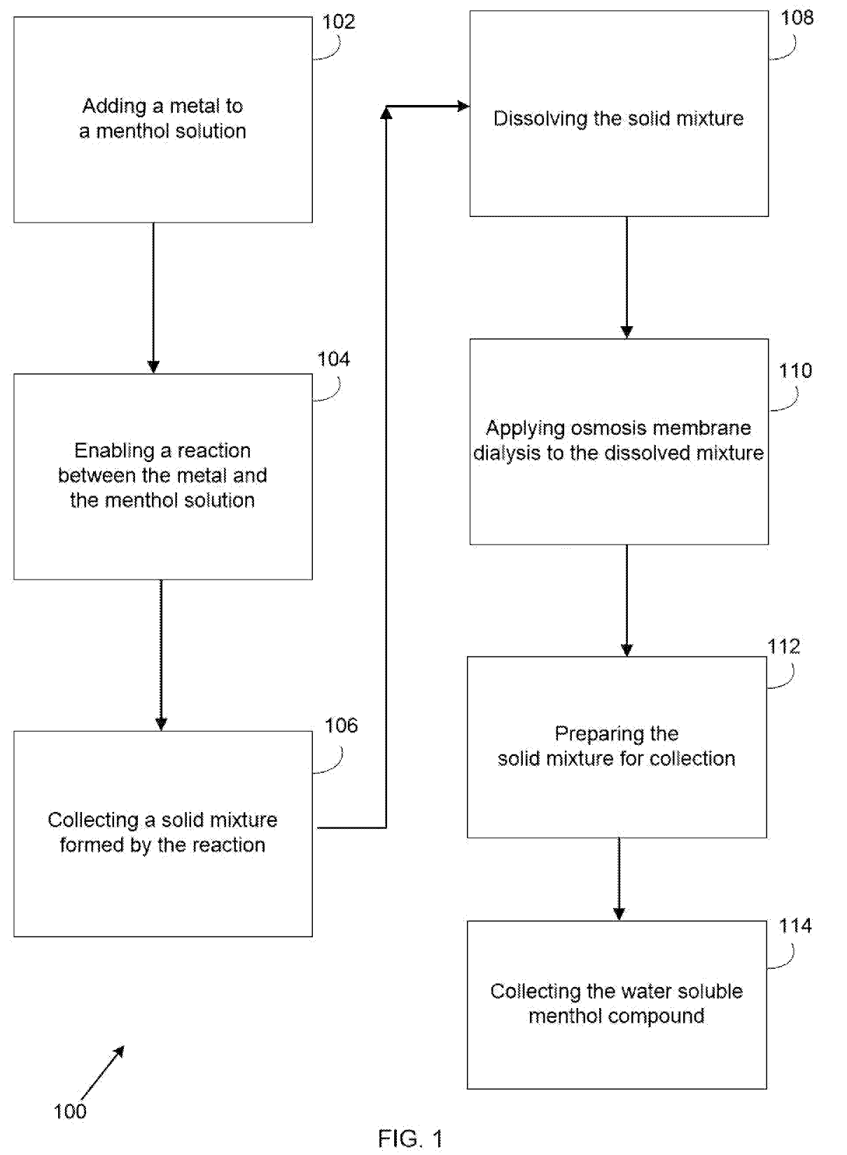

FIGS. 1-3 illustrate the process, molecular structures, and experimental data of the production of a menthol compound 200. A method 100 for producing water soluble menthol compound 200 is disclosed that produces a menthol compound 200 that is soluble in alcohol and ether, yet insoluble in water. The menthol compound 200 provides antibacterial, anti-inflammatory, and bacteriostatic effects when combined with other compounds to produce numerous medical and consumable compositions, including, without limitation, eye drops, mouth wash, juices, and noodles. In some embodiments, the produced menthol compound 200 is a menthol water-soluble sodium salt or sylvite. However in other embodiments, the menthol compound 200 may include a menthol crystal configuration.

FIG. 1 illustrates a flowchart diagram of an exemplary method 100 for producing the water soluble menthol compound 200. In one embodiment, the method 100 for producing a menthol compound 200 comprises a first Step 102 of adding a metal to a menthol solution. In some embodiments, the metal is a sodium or potassium metal. The type of metal is dependent on the desired byproduct. For example, the production of eye drops requires a sodium metal, while the production of mouth wash requires the use of a potassium metal.

The water soluble menthol compound 200 may include a water soluble sodium salt or sylvite of menthol. The menthol solution is used for washing, and may include ethereal solution or alcohol solution. The ethereal solution is diethyl ether or diethylene oxide. The alcohol solution is an ethanol. Additionally, Step 102 of adding the metal to the menthol solution forms a mole ratio of about 1:1.1 to 1.3, depending on the desired byproduct composition. For example, the production of eye drops requires a mole ratio of 1:1.1, while the production of mouth wash requires a mole ratio of 1:1.2. The metal is added to the menthol compound 200 under a nitrogen atmosphere. Since, in this method 100, oxidation is not desired in the reaction between the metal and the menthol solution, the nitrogen atmosphere serves as an inert replacement for air.

A second Step 104 comprises enabling a reaction between the metal and the menthol alcohol solution. In some embodiments, enabling the reaction between the metal and the menthol alcohol solution includes stirring the reaction mixture between about 24° to 28° Celsius for about 500 to 700 minutes. The stirred solution is then rested overnight to hydrate into a solid mixture. The method 100 may further include a Step 106 of collecting a solid mixture formed by the reaction. The Step 106 of collecting the solid mixture includes filtering, drying, condensing, and washing the solid mixture to remove traced of to remove traces of moisture from the solution.

Those skilled in the art will recognize that thoroughly washing the precipitate is necessary to remove all adsorbed species which will add to weight of the solid mixture. It is also less advised to use water as a washing solution since peptization would occur. Therefore, the method 100 utilizes ethereal solution or alcohol solution for washing the solid mixture. However, in alternative embodiments, dilute nitric acid, ammonium nitrate, or dilute acetic acid may be used. A quantitative determination of the menthol alcohol may be determined based on the mass of the solid mixture. Techniques such as gravimetric analysis or vaporization with a cryogenic trap may be employed for this purpose.

In some embodiments, a Step 108 comprises dissolving the solid mixture. The solid mixture can be dissolved with water to reform into a liquid. The dissolved mixture in the liquid form may then be separated further though osmosis membrane dialysis. A Step 110 may include applying osmosis membrane dialysis to the dissolved mixture. The dissolved mixture passes through a selectively permeable membrane to separate the final water soluble menthol compound 200 from impurities. Because of the selectively permeable membrane, nothing but water and other very small particles, can be diffused through osmosis. In one embodiment, the osmosis membrane dialysis comprises dialysis tubing in which movement through the membrane occurs from a high concentrate to a lower concentrate.

The method 100 may also include a Step 112 of preparing the solid mixture for collection. The preparation may include decompressing the solid mixture and removing water from the solid mixture. The water is removed to form a final solid, such as a crystalline or salt. A final Step 114 comprises collecting the water soluble menthol compound 200.

The produced menthol compound 200 may include an organic compound derived from mentha arvensis oil, and extracted from mint leaves and stems. In one embodiment, the menthol compound 200 comprises a molecular formula of C₁₀H₂₀O. The menthol compound 200 appears in molten or lump form. In some embodiments, the menthol compound is in the form of crystals that appear colorless, prism-shaped, or needle shaped. In another embodiment, the menthol compound 200 is a white color crystal. The crystals are generally solid at about 42° Celsius, melting at a temperature slightly above 43° Celsius.

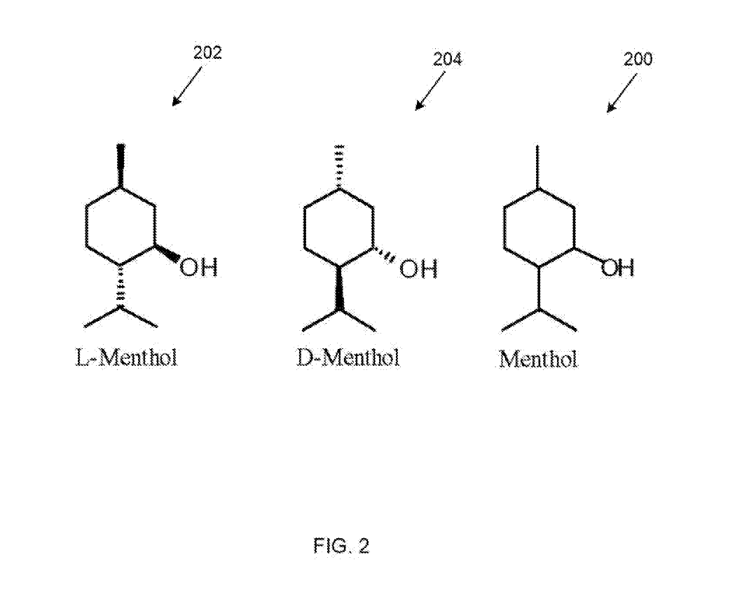

As referenced in FIG. 2, the method 100 produces various isomers of menthol, including, without limitation, a menthol compound 200, L-menthol 202, D-menthol 204, DL-menthol, and racementhol. In some embodiments, the menthol compound 200 may be used in the preparation of perfumes, cosmetics, analgesic balms, ointments, lotions, creams, tooth paste, shampoos and conditioners. Those skilled in the art will recognize that menthol compound 200 is effective as an anti-bacterial and anti-fungal agent. Additionally, the menthol crystals produced by the method 100 can be used in perfumes, cosmetics, analgesic balms, ointments, lotions, creams, tooth paste, shampoos and conditioners. Additionally, menthol crystals may also be used as a cooling agent. The method 100 produces isomers of menthol, which may be used as excitants in medications; applied on skin or mucous membrane to cool and relieve itches; and be orally administered to act as a carminative for relieving headaches, rhinitis, pharyngitis, and laryngitis. The ester of the menthol 200 is used in spices and medications, as well as a high grade perfume.

However, in one alternative embodiment, the method 100 is used for the production of a water soluble borneol, which has a minty flavor and a similar molecular as the water soluble menthol compound 200. The water soluble borneol that is formed comprises a white, translucent, lumpy, very slightly water-soluble, solid terpene alcohol. The molecular structure of borneol—C₁₀H₂₀O—is similar to the menthol compound formula of C₁₀H₂₀O, and occurs in various isomeric forms. Similar to the menthol compound, the water soluble borneol also has anti-bacterial properties and is known to have a burning, mint like taste. It is also significant to note that since borneol easily oxidizes to a ketone camphor, the nitrogen atmosphere inhibits this undesirable effect. Rather, the l-borneol isomer is chiefly produced. In some embodiments, the produced water soluble borneol may be used in perfume and in the manufacture of organic esters.

In some embodiments, the produced menthol compound 200 may be combined with additional compounds to produce eclectic medical and consumable compositions, including, without limitation, eye drops, antibacterial agents for washing the mouth, juices, and noodles. In a first embodiment, the method 100 produces a water soluble menthol sodium salt for use in eye drop solutions. The preparation of the menthol sodium salt occurs under a nitrogen atmosphere. In a reaction vessel sodium metal is added to the menthol alcoholic solution. The mole ratio of menthol to sodium metal is 1:1.1. The mixture is stirred slowly for 600 minutes at the temperature of 25° Celsius. The full reaction is reached after setting overnight. The subsequent reaction mixture is obtained, filtered, condensed, washed with ethanol, dried, and the solid mixture is collected. The solid is dissolved in water and osmosis membrane dialysis is applied for 5 days. The mixture is then decompressed and water is removed therefrom to collect a menthol sodium salt.

The water soluble menthol sodium salt is combined with additional compounds to form the eye drop composition. The compounds, in percentage, may include, without limitation: Mitomycin C 0.06%; Poloxamer P407 30%; Poloxamer P188 11%; Carbomer 0.2%, menthol sodium salt 5%; and normal saline 53.74%. The method 100, in this embodiment, produces menthol sodium salt that can be applied not only to eye drops, it can also be applied to the externally applied skin medications and oral medications of the pharmaceutical field. It mostly has an antibacterial and anti-inflammatory effect. From the current raw materials during the production, only about 0.0001 to 0.5% out of the total raw materials of the menthol salt is needed.

In a second embodiment, the method 100 produces a water soluble menthol sylvite for use as an antibacterial agent, such as mouth wash. The preparation of the menthol sodium salt occurs under a nitrogen atmosphere. In a reaction vessel sodium metal is added to the menthol alcoholic solution. The mole ratio of menthol to sodium metal is 1:1.2. The mixture is stirred slowly for 500 minutes at the temperature of 24° Celsius. The full reaction is reached after setting overnight. The subsequent reaction mixture is obtained, filtered, condensed, washed with ethanol, dried, and the solid mixture is collected. The solid is dissolved in water and osmosis membrane dialysis is applied for 3 days. The mixture is then decompressed and water is removed therefrom to collect the water soluble menthol sylvite.

The water soluble menthol sylvite is combined with additional compounds to form the antibacterial agent. The compounds, in percentage, may include, without limitation: Perilla oil 2%; water soluble menthol sylvite 5%; Ethanol 10%; Calcium hydrogen phosphate anhydrous salt 2%; Sorbitol 1%; Xylitol 2%; Ammonium Hexafluorosilicate 0.01%; and marginal distilled water. The resultant antibacterial agent that is an effective bacteriostatic that inhibits bacteria from reproducing, while not necessarily harming the bacteria otherwise. The method 100, in this embodiment, produces water soluble menthol sylvite that can be applied not only in mouth wash, but also can be applied in the field of daily chemical products such as toothpaste, detergent powder, hand soap, cleanser, air freshener, floral water, perfume, shampoo, and facial cleanser. During the production, out of the totally weight of the raw materials, only about 0.00001 to 0.5% of the water soluble menthol sylvite is needed for the regular formula. The main purpose is to achieve the antibacterial, anti-inflammatory, and bacteriostatic effect.

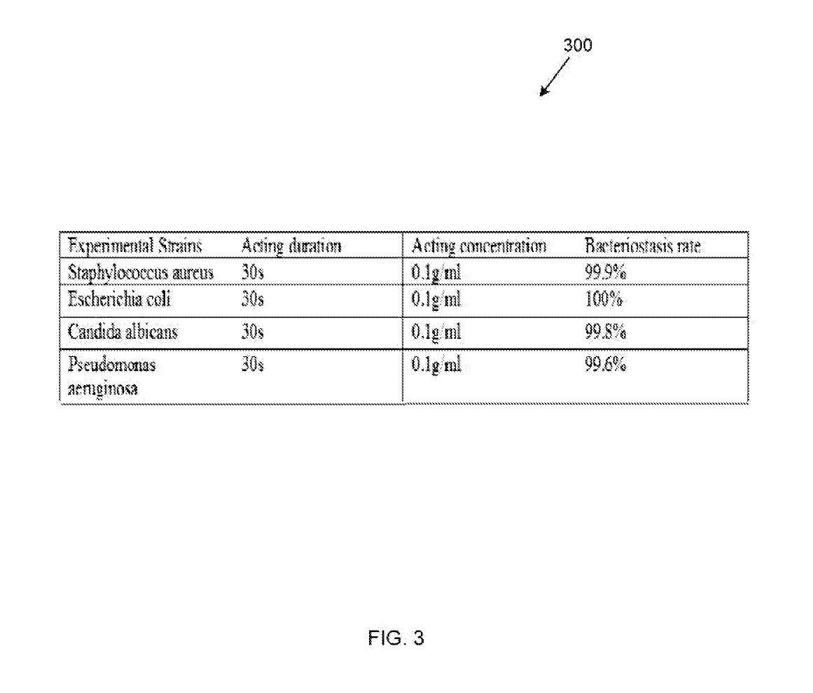

FIG. 3 illustrates a table demonstrating an antibacterial and bacteriostatic efficacy exam 300 based on the antibacterial agent produced from this embodiment of the present invention. The experimental strains of bacteria include: Staphylococcus aureus, Escherichia coli, Candida albicans, and Pseudomonas aeruginosa. Growth of the bacteria is at least partially inhibited by the water soluble menthol sylvite at a bacteriostasis rate of at least 99.6%. The duration and concentration of use is also factored into the bacteriostasis rate.

In a third embodiment, the method 100 produces a water soluble menthol sodium salt for use in a juice beverage. The preparation of the menthol sodium salt occurs under a nitrogen atmosphere. In a reaction vessel sodium metal is added to the menthol alcoholic solution. The mole ratio of menthol to sodium metal is 1:1.3. The mixture is stirred slowly for 700 minutes at the temperature of 28° Celsius. The full reaction is reached after setting overnight. The subsequent reaction mixture is obtained, filtered, condensed, washed with ethanol, dried, and the solid mixture is collected. The solid is dissolved in water and osmosis membrane dialysis is applied for 6 days. The mixture is then decompressed and water is removed therefrom to collect a menthol sodium salt.

The water soluble menthol sodium salt is combined with additional compounds to help flavor the juice. The compounds, in percentage, may include, without limitation: juice 99% and menthol sodium salt 0.1%. The method 100, in this embodiment, produces the water soluble menthol sodium salt that can be applied not only in juices, but also in the field of beverages, such as mineral water, carbonated beverages, functional beverages, and dairy beverages. During the production, out of the totally weight of the raw materials, only about 0.00001 to 0.5% of the water soluble menthol sodium salt is needed for the regular formula. The main purpose is to achieve the antibacterial, anti-inflammatory, and bacteriostatic effects.

In a fourth embodiment, the method 100 produces a water soluble menthol sodium salt for use in noodles. The preparation of the menthol sodium salt occurs under a nitrogen atmosphere. In a reaction vessel sodium metal is added to the menthol alcoholic solution. The mole ratio of menthol to sodium metal is 1:1.3. The mixture is stirred slowly for 700 minutes at the temperature of 28° Celsius. The full reaction is reached after setting overnight. The subsequent reaction mixture is obtained, filtered, condensed, washed with ethanol, dried, and the solid mixture is collected. The solid is dissolved in water and osmosis membrane dialysis is applied for 6 days. The mixture is then decompressed and water is removed therefrom to collect a menthol sodium salt. The water soluble menthol sodium salt is combined with additional compounds to help flavor the noodles. The compounds, in percentage, may include, without limitation: menthol sodium salt in the amount of 0.001% of flour.

The method 100, in this embodiment, produces menthol sodium salt that can be applied not only in noodles, but also in the field of food products, such as cakes, and cookies. During the production, out of the totally weight of the raw materials, only about 0.00001 to 0.5% of the menthol sodium salt is needed for the regular formula. The main purpose is to achieve the antibacterial, anti-inflammatory, and bacteriostatic effects.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method for producing water soluble menthol, the method comprising:
adding an alkali metal to a menthol solution;
enabling a reaction between the metal and the menthol solution;
collecting a solid mixture formed by the reaction;
dissolving the solid mixture;
applying osmosis membrane dialysis to the dissolved mixture;
preparing the solid mixture for collection; and
collecting the water soluble menthol compound.

2. The method of claim 1, wherein the water soluble menthol compound is a water soluble sodium salt or sylvite of menthol.

3. The method of claim 2, wherein the step of adding a metal to a menthol solution occurs in a nitrogen atmosphere.

4. The method of claim 3, wherein the step of adding a metal to a menthol solution forms a mole ratio of about 1:1.1 to 1.3.

5. (canceled)

6. The method of claim 4, wherein the menthol solution is contains an ethereal solution or alcohol solution.

7. The method of claim 6, wherein the ethereal solution is diethyl ether or diethylene oxide.

8. The method of claim 7, wherein the alcohol solution is ethanol.

9. The method of claim 8, wherein the step of enabling a reaction between the metal and the menthol alcohol solution includes stirring the reaction mixture between about 24° to 28° Celsius for about 500 to 700 minutes.

10. The method of claim 9, wherein the step of enabling a reaction between the metal and the menthol alcohol solution includes resting the metal and the menthol alcohol solution mixture overnight.

11. The method of claim 10, wherein the step of collecting a solid mixture formed by the reaction includes filtering, drying, condensing, and washing the solid mixture.

12. The method of claim 11, wherein an ethereal solution or alcohol solution are used for washing the solid mixture.

13. The method of claim 12, wherein the step of applying osmosis membrane dialysis to the dissolved mixture takes about 3 to 6 days.

14. The method of claim 13, wherein 0.00001% to 0.5% by weight of the water soluble menthol compound is combined with additional compounds to form beverages and food products.

15. The method of claim 14, wherein 0.00001% to 0.5% by weight of the water soluble menthol compound is combined with additional compounds to form an antibacterial agent.

16. The method of claim 15, wherein the method produces water soluble borneol.

17. A method for producing water soluble menthol salt for production of an eye drop composition, the method comprising:
adding a sodium metal to a menthol solution, the mole ratio of menthol solution to sodium metal about 1:1.1;
enabling a reaction between the metal and the menthol solution, the reaction including stirring a reaction mixture for about 600 minutes at about 25° Celsius;
collecting a solid mixture formed by the reaction;
dissolving the solid mixture;
applying osmosis membrane dialysis to the dissolved mixture for at least 5 days;
decompressing the solid mixture to remove water; and
collecting the water soluble menthol salt.

18. The method of claim 17, wherein the eye drop composition comprises the following:
Mitomycin C 0.06%; Poloxamer P407 30%; Poloxamer P188 11%;
Carbomer 0.2%, water soluble menthol salt 5%, and normal saline 53.74%.

19. A method for producing water soluble menthol sylvite for production of an antibacterial agent, the method comprising:
adding potassium to a menthol solution, the mole ratio of menthol solution to potassium metal about 1:1.2;
enabling a reaction between the metal and the menthol solution, the reaction including stirring a reaction mixture for about 500 minutes at about 24° Celsius;
collecting a solid mixture formed by the reaction;
dissolving the solid mixture;
applying osmosis membrane dialysis to the dissolved mixture for at least 3 days;
decompressing the solid mixture to remove water; and
collecting the water soluble menthol sylvite.

20. The method of claim 19, wherein the antibacterial agent comprises the following:
Perilla oil 2%; water soluble menthol sylvite 5%; Ethanol 10%; Calcium hydrogen phosphate anhydrous salt 2%; Sorbitol 1%; Xylitol 2%;
Ammonium Hexafluorosilicate 0.01%; and marginal distilled water.
